(19) United States
(12) Patent Application Publication
(10) Pub. No.: US 2007/0197815 A1
(43) Pub. Date: Aug. 23, 2007

(54) PROCESS FOR THE PREPARATION OF AN ALKANEDIOL AND A DIALKYL CARBONATE

(76) Inventors: Evert Van Der Heide, Amsterdam (NL); Timothy Michael Nisbet, Amsterdam (NL)

Correspondence Address: SHELL OIL COMPANY, P O BOX 2463, HOUSTON, TX, 772522463, US

(21) Appl. No.: 11/678,003

(22) Filed: Feb. 22, 2007

(30) Foreign Application Priority Data
Feb. 22, 2006 (EP) 06110249.7

(51) Int. Cl. C07C 69/96

(52) U.S. Cl. 558/277

(57) ABSTRACT

A process for the preparation of an alkanediol and a dialkyl carbonate is provided, which process comprises: (a) contacting an alkylene carbonate with an alkanol feedstock under transesterification conditions in a reactive distillation column to obtain an upwardly moving stream comprising dialkyl carbonate and the alkanol and a downwardly moving stream comprising the alkanediol; (b) recovering the alkanediol at the bottom of the column; (c) withdrawing a dialkyl carbonate- and alkanol-containing product stream at the upper part of the column, which upper part is below the top of the column; and (d) removing lower-boiling compounds at the top of the column.

This application claims the benefit of European Patent Application 06110249.7, filed Feb. 22, 2006 which is incorporated herein by reference.

### FIELD OF THE INVENTION

The present invention relates to a process for the preparation of an alkanediol and a dialkyl carbonate. More particularly, the invention relates to a process for the preparation of such compounds from an alkylene carbonate and an alkanol.

### BACKGROUND

A process for the preparation of a dialkyl carbonate is known from e.g. U.S. Pat. No. 5,231,212. This document discloses a process for the continuous preparation of dialkyl carbonates by transesterification of an alkylene carbonate with alcohols in the presence of a catalyst in a reaction column. The reactants are passed in counter-current such that the alkylene carbonate is metered into the upper part of the column and the alcohol is metered into the lower part of the column. The catalyst is either arranged as a fixed bed or also metered into the upper part of the column in solution or suspension. The dialkyl carbonate formed, if appropriate as a mixture with alcohol, is removed at the top of the column and the alkane diol is removed at the foot of the column. If the catalyst is provided as a solution or a suspension the catalyst is also removed at the bottom of the column.

In the known process it is indicated that the bottoms stream withdrawn at the foot of the column may contain some contaminants, e.g., the alcohol and alkylene carbonate. The known process does not address the problem of the build up of low-boiling by-products. Such by-products can for instance be carbon dioxide that may be formed due to the hydrolysis of alkylene carbonate by small amounts of water that may be present in the alkanol or any other starting material. Other by-products that may be formed include acetaldehyde, propionaldehyde and acetone. Whereas the alkylene carbonate is generally produced from alkylene oxide and carbon dioxide, the alkylene carbonate feed may contain some alkylene oxide. Also other gases, such as nitrogen, may be entrained by the reactants.

### SUMMARY OF THE INVENTION

On an industrial scale it is very important that the content of by-products in the reaction products is kept as low as possible. Although the known process provides reasonably pure products, it has now been found that a purer product can be obtained if the dialkyl carbonate product is not removed at the top of the column, but at an upper part of the column.

Accordingly, the present process provides a process for the preparation of an alkanediol and a dialkyl carbonate comprising:
(a) contacting an alkylene carbonate with an alkanol feedstock under transesterification conditions in a reactive distillation column to obtain an upwardly moving stream comprising dialkyl carbonate and the alkanol and a downwardly moving stream comprising the alkanediol;
(b) recovering the alkanediol at the bottom of the column;
(c) withdrawing a dialkyl carbonate- and alkanol-containing product stream at the upper part of the column, which upper part is below the top of the column; and
(d) removing lower-boiling compounds at the top of the column.

### DETAILED DESCRIPTION

The process of the present invention includes the transesterification of an alkylene carbonate with an alkanol. This transesterification reaction is known, as is apparent from e.g., U.S. Pat. No. 5,231,212 and U.S. Pat. No. 5,359,118. The starting materials of the transesterification are preferably selected from C₂-C₆ alkylene carbonate and C₁-C₄ alkanols. More preferably the starting materials are ethylene carbonate or propylene carbonate and methanol, ethanol or isopropanol. The most preferred alkanols are methanol and ethanol.

The transesterification step is advantageously carried out in a column into which the alkylene carbonate is fed at the upper part, such that the alkylene carbonate flows down in counter current contact with upwardly moving alkanol. The product of the reaction is a dialkyl carbonate and an alkanediol. The dialkyl carbonate is recovered at the upper part of the column. The alkanediol is recovered as the bottom stream.

The transesterification is suitably conducted in the presence of a catalyst. Suitable catalysts have been described in U.S. Pat. No. 5,359,118 and include hydrides, oxides, hydroxides, alcoholates, amides, or salts of alkali metals, i.e., lithium, sodium, potassium, rubidium and caesium. Preferred catalysts are hydroxides or alcoholates of potassium or sodium. It is advantageous to use the alcoholate of the alkanol that is being used as feedstock. Such alcoholate can be added as such or formed in situ.

Other suitable catalysts are alkali metal salts, such as acetates, propionates, butyrates, or carbonates. Further suitable catalysts are described in U.S. Pat. No. 5,359,118 and the references mentioned therein, e.g., EP-A 274 953, U.S. Pat. No. 3,803,201, EP-A 1082, and EP-A 180 387.

The transesterification conditions are known in the art and suitably include a temperature from 40 to 200° C., and a pressure from 50 to 400 kPa. Preferably, the pressure is close to atmospheric. The temperature depends on the alkanol feedstock and pressure used. The temperature is kept such that it is close to and above the boiling point of the alkanol, e.g. up to 5° C. above the boiling point. In the case of methanol and atmospheric pressure, the temperature is close to and above 65° C., for instance between 65 and 70° C.

The transesterification reaction is advantageously conducted in a column furnished with internals, like a distillation column. Hence, it may contain trays with bubble caps, sieve trays, or Raschig rings. The skilled person will realise that several packings and tray configurations will be possible. It is within his skill to determine the theoretical trays in such columns. The alkylene carbonate will be fed at the upper part of such a column and will flow down. Surprisingly, it has been found that an even purer dialkyl carbonate product stream can be obtained when the alkylene carbonate is fed into the column at a position above the position from which the dialkyl carbonate product stream is withdrawn. The distance between the position at which alkylene carbonate is fed into the column and the position at which the product stream is withdrawn suitably ranges from 1 to 10 theoretical trays.

The alkylene carbonate will generally have a higher boiling point than the alkanol. In the case of ethylene and propylene carbonate the atmospheric boiling points are above 240° C. The alkylene carbonate will flow down over the trays or rings and brought into contact with the alkanol that flows upwardly. When the transesterification catalyst is homogeneous, such as an alkali metal alcoholate, it is also introduced in the upper part of the column. The alkanol feedstock is introduced at a lower point. The feedstock may be completely vaporous. However, it is also possible to introduce the feedstock into the column partly in the liquid phase. It is believed that the liquid phase ensures a higher concentration of alkanol in the lower part of the column with a beneficial effect on the overall transesterification. It is distributed over the width of the column via the inlet and the column internals. The ratio between the vaporous and the liquid part of the alkanol feedstock may be varied between wide ranges. The vapour/liquid weight ratio is suitably from 1:1 to 10:1 wt/wt.

The person skilled in the art will know that the transesterification is an equilibrium reaction. Therefore, he shall suitably employ an excess of the alkanol. The molar ratio of alkanol to alkylene carbonate is suitably from 5:1 to 25:1, preferably from 6:1 to 15:1, more preferably from 7:1 to 9:1. The amount of catalyst can evidently be much smaller. Suitable amounts include from 0.1 to 5.0% wt based on alkylene carbonate, preferably from 0.2 to 2% wt.

The reactive distillation results in an upwardly moving stream containing the dialkyl carbonate and any excess unreacted alkanol, and a downwardly moving stream containing the alkane diol and the catalyst that are recovered at the bottom of the column. Due to some water that may be contained in the alkanol some hydrolysis of the alkylene carbonate may take place, forming alkanediol and carbon dioxide. Other lower-boiling by-products or contaminants can be aldehydes, ketones and alkylene oxides, and gases entrained with the reactants, such as nitrogen. In this specification by lower-boiling compounds are understood compounds that have lower boiling point than the alkanol.

The alkanediol stream recovered at the bottom is suitably subjected to a separation of the alkanediol. Thereto, the bottom stream is split suitably in a fractionation column into a catalyst-rich stream and a stream comprising the alkanediol and, optionally, some alkanol. After optional purification, e.g., by further distillation, the alkanediol is recovered as eventual product. The catalyst-rich stream is suitably recycled to the reactive distillation zone. Also any alkanol that is separated from the bottom stream can be recycled.

The upwardly moving stream is withdrawn at a position below the top of the column. Due to the distillation action that occurs in the column a significant portion of the lower-boiling by-products is separated between the top of the column and the position below at which the dialkyl carbonate- and alkanol-containing stream is withdrawn. The lower-boiling by-products are removed at the top of the column. The distance between the top and the position at which the product is withdrawn suitably ranges from 1 to 10 theoretical trays.

The product stream with dialkyl carbonate and alkanol is suitably subsequently separated into an alkanol-rich stream and a dialkyl carbonate-rich stream. This can suitably be done by distillation. However, as indicated in U.S. Pat. No. 5,359,118 many alkanols and their corresponding dialkyl carbonates form azeotropes. Therefore simple distillation may not be sufficient to achieve a satisfactory separation. Therefore it is preferred to use an extractant to facilitate the separation between the dialkyl carbonate and the alkanol. The extractant can be selected from many compounds, in particular alcohols such as phenol, or anisole. However, it is preferred to employ an alkylene carbonate as extractant. It is most advantageous to obtain the separation in the presence of the alkylene carbonate that is being used as starting material for the eventual alkanediol.

The extractive distillation is preferably conducted in two columns. In the first column separation is achieved between the alkanol and a dialkyl carbonate/alkylene carbonate mixture. In the second column the separation between the dialkyl carbonate and the alkylene carbonate is achieved. The alkylene carbonate is suitable recycled to the first column for renewed use as extractant. The ratios between alkylene carbonate and alkanol and alkylene carbonate and dialkyl carbonate can be varied between wide ranges. Suitable ranges include from 0.2 to 2 moles of alkylene carbonate per mole of the sum of alkanol and dialkyl carbonate, preferably from 0.4 to 1.0 mole per mole.

The distillation conditions for this separation can be selected within wide ranges, as the skilled person will realize. Pressures may suitably range from 5 to 400 kPa, and temperatures from 40 to 200° C. In view of the stability of alkylene carbonate the temperature is advantageously below 180° C., whereas the lower temperature is determined by the boiling point of the alkanol. When two distillation columns are used, it is preferred to conduct the separation between alkanol and dialkyl carbonate/alkylene carbonate mixture at a higher pressure, such as 60 to 120 kPa, and the second separation between dialkyl carbonate and alkylene carbonate at lower pressure, such as 5 to 50 kPa. This will allow a sufficiently low temperature to retain a satisfactory stability for the alkylene carbonate and an efficient separation between the carbonate compounds. The dialkyl carbonate obtained is recovered as product, optionally after further purification. This further purification may comprise a further distillation step or an ion-exchange step, as described in U.S. Pat. No. 5,455,368.

The alkanol-rich stream that is obtained from the distillation of the product from the reactive distillation column is suitably recycled to the reactive distillation zone. Therefore, the alkanol feedstock comprises advantageously make-up pure alkanol and at least part of this alkanol-rich stream. This stream may be liquid and/or vaporous. The recycle stream may be mixed with the make-up pure alkanol, and subsequently be introduced into the reactive distillation zone as the alkanol feedstock. However, it is preferred to introduce the make-up alkanol into the reactive distillation zone below the introduction of the recycle stream. Thereby the advantages described in U.S. Pat. No. 5,359,118 are being obtained.

The process of the present invention can be employed for a variety of feedstocks. The process is excellently suited for the preparation of ethylene glycol, propylene glycol, dimethyl carbonate and/or diethyl carbonate. The process is most advantageously used for the production of propylene glycol (1,2-propane diol) and dimethyl carbonate from propylene carbonate and methanol.

The invention will be illustrated by the following examples.

### EXAMPLES

### Comparative Example A

A reactive distillation column has 40 theoretical trays. Propylene carbonate (5897 kg/h), is fed at tray 2. A solution of a homogeneous catalyst is fed at tray 2. Reaction takes place on all trays below the catalyst feed. Methanol vapour (16834 kg/h) is fed at tray 35. Monopropylene glycol product is removed from the bottom of the column. A mixture of dimethyl carbonate and methanol is removed at the top of the column. A reflux ratio of 0.75 mole/mole is applied. Lower-boiling compounds present in the feeds or formed in the column are as follows: nitrogen 5 kg/h, Co₂ 50 kg/h, propylene oxide 22 kg/h.

The table below shows the composition of the product stream.

### Example 1

A reactive distillation column has 45 theoretical trays. Propylene carbonate (5897 kg/h) is fed at tray 7. A solution of a homogeneous catalyst is fed at tray 7. Reaction takes place on all trays below the catalyst feed. Methanol vapour (16834 kg/h) is fed at tray 40. Monopropylene glycol product is removed from the bottom of the column. A mixture of dimethyl carbonate and methanol is removed as a vapour side draw at tray 6. Condenser duty is essentially the same as in comparative example 1. A small vapour stream is removed at the top of the column via which 32 kg/h lower-boiling compounds are removed. The vapour stream contains about 5 kg/h dimethyl carbonate and 10 kg/h methanol. Lower-boiling compounds present in the feeds or formed in the column are as follows: nitrogen 5 kg/h, CO₂ 50 kg/h, propylene oxide 22 kg/h.

The Table shows that the product stream contains substantially less lower-boiling compounds in this example than in the comparative example A.

### Example 2

The same column as in Example 1 is used. The process of Example 1 is repeated, with the exception that propylene carbonate (5897 kg/h) is fed at tray 2 instead of tray 7. Lower-boiling compounds present in the feeds or formed in the column are as follows: nitrogen 5 kg/h, CO₂ 50 kg/h, propylene oxide 22 kg/h. The small vapour stream that is removed at the top of the column contains about 31 kg/h lower-boiling compounds, about 2 kg/h dimethyl carbonate and about 12 kg/h methanol.

The Table below shows that the product stream contains substantially less light ends than in the comparative example A. Compared to example 1, less of the desired product dimethyl carbonate is lost in the top vapour stream that contains the major part of the lower-boiling compounds.

TABLE
Composition of the product stream

| | Lower-boiling | Dimethyl | Methanol, |
|---|---|---|---|
| Example | compounds, kg/h | carbonate, kg/h | kg/h |
| A | 77 | 5206 | 12638 |
| 1 | 46 | 5200 | 12628 |
| 2 | 47 | 5203 | 12626 |

1. A process for the preparation of an alkanediol and a dialkyl carbonate comprising:
(a) contacting an alkylene carbonate with an alkanol feedstock under transesterification conditions in a reactive distillation column to obtain an upwardly moving stream comprising dialkyl carbonate and the alkanol and a downwardly moving stream comprising alkanediol;
(b) recovering the alkanediol at the bottom of the column;
(c) withdrawing a dialkyl carbonate and alkanol-containing product stream at an upper part of the column, which upper part is below the top of the column; and
(d) removing lower-boiling compounds at the top of the column.

2. A process according to claim 1, in which the distance between the top and the upper part at which the product stream is withdrawn ranges from 1 to 10 theoretical trays.

3. A process according to claim 1, in which the dialkyl carbonate and alkanol-containing product stream is separated into an alkanol-rich stream and a dialkyl carbonate-rich stream.

4. A process according to claim 1, in which alkylene carbonate is fed into the column at a position above the upper part at which the dialkyl carbonate and alkanol-containing product stream is withdrawn.

5. A process according to claim 4, in which the distance between the position at which alkylene carbonate is fed into the column and the upper part at which the product stream is withdrawn ranges from 1 to 10 theoretical trays.

6. A process according to claim 1, in which the alkylene carbonate is propylene carbonate and the alkanol is methanol.
